(19) United States
(12) Patent Application Publication
(10) Pub. No.: US 2010/0010549 A1
(43) Pub. Date: Jan. 14, 2010

(54) DEVICE AND METHOD OF MINIMALLY INVASIVE EXTRACAPSULAR LIGAMENTOUS AUGMENTATION FOR CANINE STIFLE LIGAMENT INJURIES

(76) Inventors: Neville Alleyne, La Jolla, CA (US); Stuart Young, Del Mar, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 12/398,110

(22) Filed: Mar. 4, 2009

Related U.S. Application Data
(60) Provisional application No. 61/034,121, filed on Mar. 5, 2008.

(51) Int. Cl. A61B 17/58

(52) U.S. Cl. 606/86 R

(57) ABSTRACT

The present invention relates to the field of veterinary medicine. In particular, methods and devices are described for augmenting ligaments of the canine stifle joint. In some embodiments, the medial or lateral collateral ligaments are augmented or replaced using an implantable device containing biodegradable matrix containing microparticles.

### CROSS-REFERENCE TO RELATED APPLICATIONS

This application claims priority to U.S. Provisional Application No. 61/034,121, entitled “A Device and Method for Minimally Invasive Extracapsular Ligamentous Augmentation for Canine Cruciate Ligament Injuries,” filed on Mar. 5, 2008, which is hereby incorporated by reference in its entirety.

### FIELD OF THE INVENTION

The present invention relates to the field of veterinary medicine. In particular, methods and devices are described for augmenting ligaments of the canine stifle joint.

### BACKGROUND

One of the most common orthopedic problems in dogs, is injury to the stifle (hind knee) joint, and in particular, the cranial cruciate ligament of the stifle joint. The canine stifle joint is a hinge joint and connects the femur, patella and tibia. Holding these bones together are the cranial and caudal cruciate ligaments, medial and lateral collateral ligaments, and the patellar tendon. The joint is bathed in synovial fluid which is contained in the joint capsule. The cruciate ligaments reside deep within the knee joint, and the cranial cruciate ligament is much more commonly injured than the other ligaments of the canine stifle. Dogs with injuries will suffer lameness, pain, and develop associated disorders.

The causes of cruciate ligament injury can be complex. In some cases, injury can be through sudden rotation of the stifle. However, other cases can develop through no apparent trauma. It is believed that the slope of the tibial plateau along with the joint forces causes the femur to translate upon the tibial plateau. This results in a classic condition known as cranial tibial thrust. This forward movement puts the cranial cruciate ligament under significant stress and can result in attenuation or rupture of the cranial cruciate ligament.

When either the caudal or cruciate ligament does attenuate or is ruptured, it can lead to joint instability, and if left untreated it will result in progressive degenerative changes within the joint. In some cases, ligament rupture can lead to articular wear of the joint secondary to the femoral condyle engaging upon the tibial plateau and causing articular “scuffing.” These forces continue to cause degeneration and weakening of secondary restraints such as the medial and lateral meniscus. The instability can lead to tears of the medial and lateral meniscus, which can cause further instability, pain and lameness. In addition, as joint changes develop, the cruciate ligaments undergo alteration in their migrostructure. Collagen fibrin become hyalinized, and the tensile strength of the ligament is reduced, making the ligament more susceptible to damage from minimal trauma.

Currently, a number of extracapsular and intra-articular surgical techniques can be used to treat stifle ligament injuries. Extracapsular techniques include imbrication of the lateral joint tissues with one or more sutures. The sutures are placed in a general anteroposterior orientation to eliminate the cranial displacement of the tibia on the femur (cranial drawer). By placing the imbrication suture or sutures on the lateral aspect of the joint, the tendency for inward rotation of the tibia due to cranial cruciate ligament insufficiency is also prevented. However, current extracapsular imbrication procedures are limited in the ability to provide sufficient stability to the cranial cruciate deficient stifle joint, and are also met with limited success in larger dogs.

Intra-articular repairs include reconstruction or replacement of the cruciate ligaments with either an autogenous or a synthetic graft. Replacement techniques involve the re-creation of an intra-articular structure in the approximate spatial orientation of the normal cranial cruciate ligament. The graft is usually passed through drill holes in the femur and tibia and, depending on the technique used, is attached to the soft tissues of the femur or tibia. Not only are such techniques invasive, but the ideal transplant material has not yet been found. Ideally, a material would possess great strength, some elasticity, and tolerate wear and tear in the joint for years, and be non-irritant.

Another intra-articular technique includes tibial plateau leveling osteotomy (TPLO) where the tibia is cut and the slope between the femur and tibia reduced. This procedure carries with it complications such, infection, nonunion, fracture of hardware, arterial and nerve injury, severe limitation in joint movement and chronic pain. Accordingly, there is a need for improved methods and devices to treat injured ligaments of the canine stifle joint.

### SUMMARY

The present invention relates to methods and devices for augmenting ligaments of the canine stifle. Some methods for augmenting the stifle joint in a canine subject can include identifying a subject in need of stifle joint augmentation, delivering an implantable device to the joint, in which the implantable device includes a biodegradable matrix and a plurality of microparticles; and contacting the implantable device with at least a portion of the joint.

In some methods, the at least a portion of the joint can include one or more sites selected from the lateral collateral ligament, medial collateral ligament, cranial cruciate ligament, caudal cruciate ligament, fascia lata, capsule, or patella tendon.

In some methods, the implantable device can include a sheet. In more methods the sheet can include fenestrations.

In some methods, the biodegradable matrix comprises bovine collagen. In more methods, the biodegradable matrix can include one or more materials selected from albumin, gelatin, chitosan, hyaluronic acid, starch, cellulose, cellulose derivatives (e.g. methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(D, L lactide), poly (D, L-lactide-co-glycolide), poly(glycolide), poly(hydroxybutyrate), poly(alkylcarbonate), poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), and copolymers thereof.

In some methods, the plurality of microparticles can include one or more materials selected from poly methacrylate, polymethyl methacrylate, hydroxapatite, powdered bone, or glass.

In some methods, the plurality of microparticles can be substantially spherical with a diameter less than 200 μm. In more methods, the plurality of microparticles can be substantially spherical with a diameter less than 100 μm.

In some methods, the implantable device can also include a bioactive agent. In more methods, the bioactive agent can include an agent selected from the group consisting of a local anesthetic, non-steroidal anti-inflammatory drug, antibiotic, and antineoplastic agent.

In some methods, the bioactive agent can comprise lidocaine.

In some methods,the implantable device further can include a substrate. In more methods, the substrate can include one or more materials selected from nylon, Dacron, or Teflon.

Some methods can also include anchoring the implantable device to one or more sites at the joint.

In some methods, the delivering can comprise an open surgical procedure.

In some methods, the delivering can comprise percutaneous delivery.

Some methods can also include performing an extra-articular procedure. In such methods, the extra-articular procedure can be selected from fascia lata imbrication, lateral retinacular imbrication, modifications of the lateral retinacular imbrication technique, or posterolateral capsulorrhaphy.

Some methods can also include performing an intra-articular technique. In such methods, the intra-articular technique can be selected from fascia lata repair, modified fascia lata repair, patella tendon repair, or over-the-top repair.

In some methods, the cranial cruciate ligament of the subject has undergone partial disruption or fill disruption.

In addition to the methods described herein, also provided are canine stifle joints including an implantable device, in which the implantable device comprises collagen and microparticles.

In some embodiments, the collagen comprises bovine collagen.

In some embodiments, the microparticles are substantially spherical with a diameter less than 200 μm. In more embodiments, the microparticles are substantially spherical with a diameter less than 100 μm.

In some embodiments, the implantable device further comprises a bioactive agent.

In more embodiments, the bioactive agent comprises an agent selected from the group consisting of a local anesthetic, non-steroidal anti-inflammatory drug, antibiotic, and antineoplastic agent. In more embodiments, the bioactive agent comprises lidocaine.

### BRIEF DESCRIPTION OF THE DRAWINGS

FIG. 1 shows a schematic of a canine stifle with an augmented medial collateral ligament.

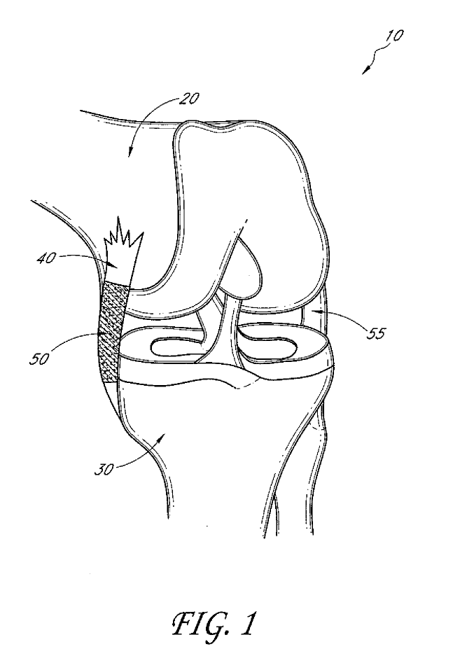

FIG. 2 shows a schematic of a canine stifle with a replaced medial collateral ligament.

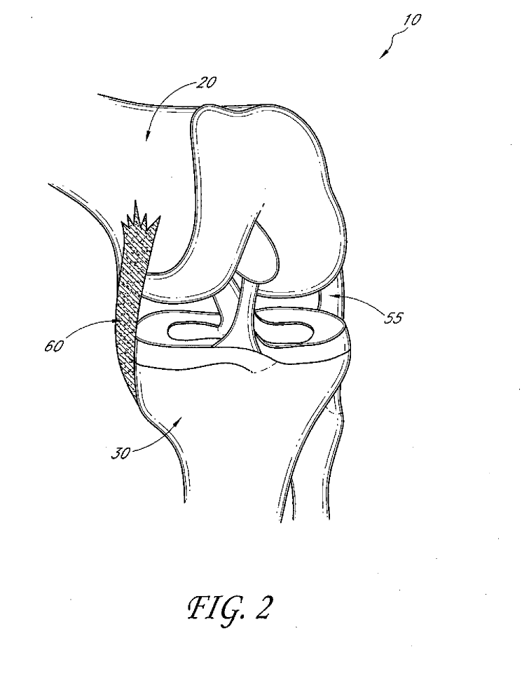

FIG. 3 shows a schematic of a canine stifle with an augmented cranial cruciate ligament.

### DETAILED DESCRIPTION

The present invention relates to methods and devices for augmenting ligaments of the canine stifle joint. In particular embodiments, methods are provided that can include identifying a subject in need of stifle joint augmentation, delivering an implantable device to the joint, in which the implantable device includes a biodegradable matrix and a plurality of microparticles; and contacting the implantable device with at least a portion of the joint.

In some embodiments, the implantable device can include a biodegradable matrix, such as collagen, and microparticles comprised of polymethyl methacrylate (PMMA). In some embodiments, the biodegradable matrix is a mesh that can provide both immediate tensile strength at a site of implantation, and a scaffold for canine fibroblasts to encapsulate the PMMA microparticles, to secrete components of the extracellular matrix, and to ultimately absorb the biodegradable collagen matrix. By stimulating the host to produce collagen and other extracellular components at a site of repair, the tensile strength of the repair can increase. In addition, the biodegradable collagen and PMMA microparticles farther invokes the host's inflammatory response to further provoke a fibrotic response to the implantable device, thus strengthening a repair further.

Some embodiments include wrapping and/or attaching a biodegradable collagen mesh containing PMMA microparticles around the medial or lateral collateral ligaments of the stifle joint. This method has the advantage of being minimally invasive, thus the veterinary surgeon can minimize her incision on the medial and lateral collateral ligament, reducing blood loss, and minimizing articular destruction, as compared more invasive methods. Moreover, wrapping a ligament with the mesh, strengthens the ligament two-fold. First, the mesh provides tensile strength to the ligament, and second, the tensile strength increases as the biodegradable collagen is replaced by the host's collagen. In addition, by instituting earlier treatment through an extracapsular approach, namely, treatment of the lateral and medial collateral ligament, degenerative changes that can occur to the articular surface, such as osteophyte formation and meniscal injury are reduced. This approach can preserve motion and stability without lengthy operations that can cause morbidity.

As will be apparent, the implantable devices, and meshes described herein can be placed anywhere in the joint where a ligament needs to be augmented, for example, sites of extracapsular repairs or lubrications, and intra-articular repairs.

The description provided herein is directed to certain specific embodiments. However, the invention can be embodied in a multitude of different ways. Reference in this specification to “one embodiment” or “an embodiment” means that a particular feature, structure, or characteristic described in connection with the embodiment is included in at least one embodiment. The appearances of the phrase “in one embodiment,” “according to one embodiment,” or “in some embodiments” in various places in the specification are not necessarily all referring to the same embodiment, nor are separate or alternative embodiments mutually exclusive of other embodiments. Moreover, various features are described which may be exhibited by some embodiments and not by others. Similarly, one or more features may be described for one embodiment which can also be reasonably used in another embodiment.

As used herein, “at least a portion” can refer to at least about 0.1%, 1%, 10%, 20%, 30%, 40%, 50%, 60%, 70%, 80,%, 90%, 99% , and 100%.

Some of the implantable devices described herein are adapted to promote a fibrotic response at a site of contact. As used herein, “fibrotic response” or “fibrosis” can refer to the formation of fibrous tissue in response to medical intervention. Implantable devices which induce a fibrotic response can do so through one or more mechanisms, for example, stimulating migration or proliferation of connective tissue cells, such as fibroblasts, smooth muscle cells, and vascular smooth muscle cells; inducing production of extracellular matrix components, such as collagen; promoting tissue remodeling; and inducing or promoting angiogenesis.

An implantable device can comprise one or more components that can include, for example, a plurality of microparticles, a biodegradable matrix, a bioactive agent, and/or a substrate. The following description provides embodiments of implantable devices and methods of using such devices.

In some embodiments, microparticles can promote a fibrotic response at the site of implantation and provide a scaffold to promote connective tissue deposition around the microparticles. Microparticles can be microspheres, and/or nanoparticles. As will be understood, microparticles may be small enough to be delivered to a site, for example, by injection, but large enough to resist phagocytosis and the lymphatic and blood system from washing away any of the microparticles. As such, microparticles can have a diameter of greater than about 10 μm. In some embodiments, the microparticles can have a diameter between about 20 μm to about 200 μm, a diameter between about 25 μm to about 100 μm, or a diameter between about 30 μm to about 50 μm. The microparticles can also be highly refined to limit any inflammation from smaller particles, and to increase the roundness and smoothness properties of the particles.

The microspheres can comprise an inert, histocompatible material, such as glass, hydroxapatite, powdered bone, or a polymer. The polymer can be cured and polymerized prior to implantation to reduce toxic or carcinogenic potential of the monomers or cure agents. The inert histocompatible polymer can be an acrylic polymer. The acrylic polymer can be a polymer of methacrylate or one of its esters, such as methyl methacrylate, ethyl methacrylate, n-butyl methacrylate, isobutyl methacrylate, lauryl methacrylate, and 2-ethylhexyl methacrylate or any combination or copolymer thereof. In preferred embodiments, microparticles can comprise polymethylmethacrylate (PMMA). Some embodiments in the form of a gel or paste are described in U.S. Pat. No. 5,344,452, which is incorporated by reference in its entirety.

Microparticles can be porous or non-porous. Porous microparticles containing an additional agent may be used to deliver agents to the site of implantation.

In some embodiments, the microparticles can be suspended in a suspension agent. The suspension agent can be an aqueous or non-aqueous solution. The suspension agent can be of sufficient viscosity to promote the suspension of the microparticles. The suspension agent can be, for example, up to about 0.1%, 0.2%, 0.5%, 1.0%, 2.0%, 5.0%, 10%, 15%, 20%, 30%, 40%, 50%, 60%, 70% and 80% by volume microparticles. The amount of microparticles used is determined in part by other components of the suspension agent, such as the carrier concentration, and the method of implantation, such as injection.

The suspension agent can also contain a polymer, which can be histocompatible, as a carrier. Such a carrier can be a biodegradable matrix. A biodegradable matrix can comprise a biodegradable polymer. Examples of biodegradable polymers include collagen, albumin, gelatin, chitosan, hyaluronic acid, starch, cellulose, cellulose derivatives (e.g. methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(D, L lactide), poly (D, L-lactide-co-glycolide), poly(glycolide), poly(bydroxybutyrate), poly(alkylcarbonate), poly(orthoesters), polyesters, poly(hydroxyvaleric acid), polydioxanone, poly(ethylene terephthalate), poly(malic acid), poly(tartronic acid), polyanhydrides, polyphosphazenes, poly(amino acids), and copolymers thereof (see generally, Ilium, L., Davids, S. S. (eds.) “Polymers in Controlled Drug Delivery” Wright, Bristol, 1987; Arshady R., “Preparation of biodegradable microspheres and microcapsules.” J. Controlled Release 17:1-22, 1991; Pitt C. G., “The controlled parenteral delivery of polypeptides and proteins.” Int. J. Pharm. 59:173-196, 1990; Holland et al, “Polymers for Biodegradable Medical Devices. 1. The Potential of Polyesters as Controlled Macromolecular Release Systems.” J. Controlled Release 4:155-180, 1986). As will be appreciated, in some embodiments, an implantable device can include microparticles suspended, mixed, embedded, or coated in a biodegradable matrix.

In preferred embodiments, the biodegradable polymer can comprise collagen. Collagen may allow for the separation of the microspheres to allow tissue ingrowth. The collagen can be in many types and forms, or in combinations thereof. For example, collagen can be Type I, II or III. Collagen can be native, denatured or cross linked. The various types and forms of collagen are described generally in Methods in Enzymol. (1982) 82:3-217, Pt. A, incorporated by reference in its entirety. For example, collagen can be produced from animal derived tissues such as bovine or porcine hides, avian combs, human tissues such as cadaver skin or human cell cultures or through recombinant methods. In some embodiments, an implantable device can contain a collagen fully dissolved or in suspension. The solution can contain up to about 0.1%, 0.2%, 0.5%, 1.0%, 2.0%, 2.5%, 3.0%, 3.5%, 4.0%, 4.5%, 5%, 6%, 7%, 8%, 9%, 10%, 20%, 30%, 40%, 50%, 60%, 70%, or 80% (v/v) collagen content. The amount of collagen content in the solution is in part determined by the resultant viscosity, the percentage of other components such as microparticles and the method of implantation, such as injection.

In particular embodiments, an implantable device comprises a collagen matrix and microparticles. An example of a commercially available material that may be used in some embodiments includes ARTEFILL (Artes Medical Inc.). ARTEFILL comprises PMMA microparticles suspended in bovine collagen.

Other examples of commercially available materials that have been used for tissue repair and cosmetic applications include bovine collagen products such as ZYDERM I, ZYDERM II, and ZYPLAST (each produced by Allergan Inc.); bioengineered human collagen products such as COSMODERM I, COSMODERM II, and COSMOPLAST (Allegan Inc.); and porcine collagen products such as EVOLENCE (Ortho-McNeil-Janssen Pharmaceuticals, Inc.). More examples of collagen products include collagen meshes such as INSTAT (Johnson & Johnson), and composite collagen meshes such as ALLODERM (Lifecell Corp.), as well as collagen sponges such as SURGIFOAM (Johnson & Johnson) and TERUDERMIS (Terumo Corp.).

Implantable devices described herein can include additional bioactive agents. Bioactive agents can include any composition that is able to invoke a biological response in a subject. A biological response can include, for example, responses to promote healing such as a fibrotic response, pain relief, or to prevent infection. Examples of bioactive agents that can induce a fibrotic response include silk, talc, chitosan, polylysine, fibronectin, bleomycin. As will be understood, in some embodiments, the microparticles can induce a fibrotic response. More examples of bioactive agents include local anesthetics (e.g. lidocaine, bupivacaine, procaine, tetracaine, dibucaine, benzocaine, p-buthylaminobenzoic acid 2-(diethylamino)ethyl ester HCl, mepivacaine, piperocaine, dyclonine, and opioids such as morphine, diamorphine, pethidine, codeine, hydrocodone, and oxycodone), non-steroidal anti-inflammatory drugs (e.g. ketoprofen, auranofin, naproxen, acetaminophen, acetylsalicylic acid, ibuprofen, phenylbutazone, indomethacin, sulindac, diclofenac, paracetamol, and diflunisal, Celecoxib, and Rofecoxib), antibiotics (e.g. clindamycin, minocycline, erythromycin, probenecid, and moxifloxacin), and antineoplastic agents. Antineoplastic agents can have antimicrobial activity at extremely low doses; examples include anthracyclines (e.g. doxorubicin and mitoxantrone), fluoropyrimidines (e.g. 5-FU), folic acid antagonists (e.g. methotrexate), podophylotoxins (e.g. etoposide), camptothecins, hydroxyureas, and platinum complexes (e.g. cisplatin). In preferred embodiments, the implantable device includes lidocaine. The concentration of lidocaine can be less than about 0.1%, 0.2%, 0.3%, 0.5%, 0.7%, 0.8%, 0.9%, 1%, and 5% by weight.

Implantable devices described herein can include a substrate. The microparticles and/or biodegradable matrix can be embedded in the substrate. In some embodiments, the microparticles and/or biodegradable matrix can coat or wrap at least a portion of the substrate. The substrate can comprise a non-biodegradable material such as, nylon, Dacron and Teflon. More examples of non-biodegradable materials that can be used with the embodiments described herein include polyamides, polyolefins (e.g. polypropylene and polyethylene), polyurethanes, polyester/polyether block copolymers, polyesters (e.g. PET, polybutyleneterephthalate, and polyhexyleneterephthalate), polyester cloth (e.g. DACRON), polyester sheeting (e.g. MYLAR; DuPont), nylon meshes, DACRON meshes (e.g. MERSILENE; Ethicon, Inc.), acrylic cloth (ORLON; DuPont), polyvinyl sponge (IVALON), polyvinyl cloth (VINYON-N), polypropylene mesh (MARLEX or BARD; CR Bard, Inc.; and PROLENE; Ethicon, Inc.), silicones, fluoropolymers (e.g. fluorinated ethylene propylene), and polytetrafluoroethylene (PTFE; e.g. TEFLON mesh and cloth; DuPont).

In some implementations, an implantable device can be a fluid, suspension, emulsion, microspheres, paste, gel, spray, aerosol, or sheet. With respect to sheets, the dimensions of a sheet can vary according to the application. Accordingly, sheets can be of varying sizes, thicknesses, geometries and densities. For applications such as ligament augmentation, the sheet can have a thickness of less than about 1 mm, 2 mm, 3 mm, 4 mm, 5 mm, and 10 mm. As will be appreciated, a sheet can be trimmed to the geometries and size appropriate to the application. In some embodiments, a sheet can be rectangular with a length and/or breadth sufficient to circumvent a ligament of the stifle joint.

For sheet material, woven structures are advantageous, as well as microporous materials. The implantable device may be fenestrated to promote infiltration by the host into the sheet. Such meshes can act as a scaffold. The fenestrations may be formed in a variety of geometric shapes and sizes. Initially, in some embodiments, a fenestrated implantable device may not be as strong as a solid sheet. However, because of the increased surface area and the potential for fibrovascular infiltration through the fenestrations, a fenestrated implantable device may ultimately be stronger than a solid sheet implant. The fenestrations may also be used to attach the implantable device to a site.

As used herein “subject” can refer to an animal that can benefit from the methods and devices described herein. As will be understood by one of skill in the art, “need” is not an absolute term and merely implies that the subject can benefit from the methods and devices described herein. In preferred embodiments, the subject is a dog.

Augmentation of a ligament of the stifle joint may prevent, reduce, repair or treat various joint disorders and injuries. Injuries amenable to the methods and compositions described herein can include, for example, injuries to a ligament of the stifle such as the cranial cruciate ligament, caudal cruciate ligament, medial collateral ligament, lateral collateral ligament, fascia lata, capsule, or patella tendon. Such injuries can lead to stifle instability. Moreover, the devices and methods described herein can also be utilized in association with other methods well known in the art to repair ligaments in the stifle, and in particular, to promote stabilization of the stifle (see generally, Johnson A. L. and Dunning D. Atlas of orthopedic surgical procedures of the dog and cat. Chapters 20-22 (2005), incorporated by reference in its entirety).

More methods that can be used in association with the methods and devices described herein can also include standard extra-articular techniques. Such techniques can include facial imbrication (where the stifle joint is stabilized by placement of three layers of large Lembert sutures in the muscle fascia and fibrous layers of the joint capsule), lateral retinacular imbrication (where the imbrication suture can be placed in the connective tissue surrounding the lateral fabella and ties with the limb in a functional position), modifications of the lateral retinacular imbrication technique, and posterolateral capsulorrhaphy (Pearson et al. “Lembert suture technique to surgically correct ruptured cruciate ligaments.” J Am Anim Hops Assoc 7:1, 1971; DeAngelis M. et al., “A lateral retinacular imbrication technique for the surgical correction of anterior cruciate ligament rupture of the dog.” J Am Vet Med Assoc (1970) 157: 79-84; Flo G L, “Modification of the lateral retinacular imbrication technique for stabilizing cruciate ligament injuries.” J Am Anim Hops Assoc 11:570, (1975); and Hohn R B, et al, “Surgical repair of ligamentous structures of the stifle joint.” In Bojrab M J (ed): Current Techniques in Small Animal Surgery. Philadelphia, Lea & Febiger, (1975), incorporated by reference in their entireties).

In addition, standard intra-articular techniques may also be used in conjunction with the methods and devices of the invention. Such techniques can include the fascia lata technique, modified fascia lata technique patella tendon technique, and the over-the-top technique (Paatsama S “Ligament Injuries of the Canine Stifle Joint: A Clinical and Experimental Study”. Master's thesis, Helsinki, 1952; Dickinson C R et al., “Repair of ruptured anterior cruciate ligament in the dog: Experience of 101 cases using a modified fascia strip technique.” J Am Vet Med Assoc 170:827, 1977; Dueland R. “A recent technique for reconstruction of the anterior cruciate ligament.” J Am Anim Hops Assoc 2: 1, 1966; and Arnoczky et al., “The over-the-top procedure. A technique for anterior cruciate ligament substitution in the dog.” J Am Anim Hops Assoc. 15:28-290, 1979, incorporated by reference in their entireties).

A subject can be identified by various methods, including, for example, by palpation, arthrotomy, x-ray, ultrasound, joint taps, and magnetic resonance imaging. Such methods can indicate stifle instability, and/or injury to the ligaments of the stifle joint.

Various methods can be used to deliver an implantable device to a subject. In some embodiments, the method of delivery can be during an open surgical procedure, microdisectomy, percutaneous procedure, and/or by injection.

In preferred embodiments, the implantable device comprises a sheet. In such embodiments, the sheet may be delivered to a site at the stifle joint during an open surgical procedure, microdisectomy, and/or percutaneous procedure. The sheet can contact at least a portion of the stifle joint at one or more sites, for example, the cranial cruciate ligament, caudal cruciate ligament, fascia lata, capsule, or patella tendon. In preferred embodiments, the sheet can contact at least a portion of the medial collateral ligament and/or lateral collateral ligament. In some embodiments, a sheet can circumvent a ligament of the stifle. That is to say, in some embodiments, the sheet can be wrapped around the length of at least a portion of a ligament.

In some embodiments, a sheet can be rolled into a tube. The tubular form helps contain biological grafts such as allografts, tendon grafts, ligament grafts, autogenous tendon grafts, autogenous ligament grafts, and xenografts. In addition, once fibrovascular infiltration occurs, graft strength and stability will be significantly increased. The tubular form may come in a variety of lengths and diameters to accommodate different ligament thicknesses and lengths. In one embodiment, a tubular mesh implant is fenestrated to allow sutures, anchors, or staples to anchor the implant into the post tissue or into the graft material.

It is also envisioned that devices described herein can be used as synthetic ligaments. Implantable devices in the form of tubes and cylinders may be used in methods to replace ligaments or a portion of a ligament. Such implantable devices can include anchors at each end to articulate into the origin and insertion of the cranial or caudal cruciate ligament. For an example method see Johnson A. L. and Dunning D. Atlas of orthopedic surgical procedures of the dog and cat. Chapters 22 (2005), incorporated by reference in its entirety. Synthetic ligaments can also be formed by wrapping a non-biodegradable substrate with the biodegradable matrices and microparticles described herein.

Implantable devices including sheets, meshes, tubes and cylinders can be attached at one or more sites at the stifle. Such sites will be apparent to a skilled artisan, and may include, the origin of a ligament, the insertion point of a ligament, and specific ligaments such as, the cranial cruciate ligament, caudal cruciate ligament, medial collateral ligament, lateral collateral ligament and capsule. Sheets can be anchored, attached and/or imbricated to the joint by various methods. Examples include the use of sutures, screws, anchors, hooks, staples, pins, and darts with methods well known in the art.

In an exemplary embodiment, a sheet can be used to wrap an imbricated repair of a caudal or cranial cruciate ligament. Such a repair would have the sheet anchored to a more proximal and distal end of the ligament as well as around the imbricated repair region. In an additional exemplary embodiment, an implantable device comprising a solid cylinder can be used to repair a resected ligament. The cylinder can be attached to the origin and insertion of the particular ligament.

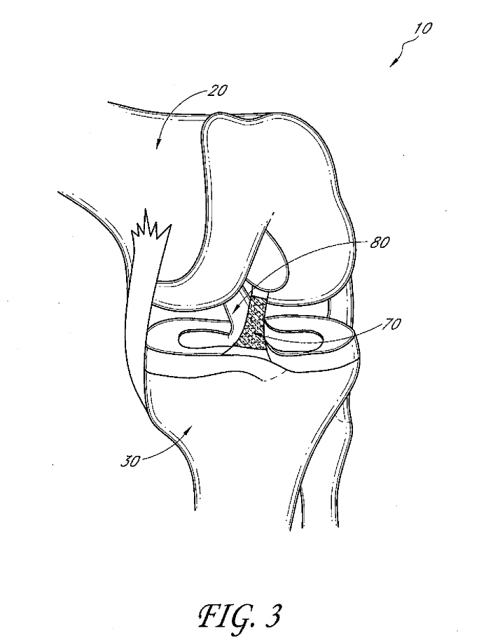

Referring to FIG. 3, the canine stifle joint (10) includes the femur (20), tibia (30) and medial collateral ligament (40). In some embodiments, an implantable device (50) comprising a collagen mesh containing PMMA microparticles can be imbricated on to the medial collateral ligament. The implantable device can contact at least a portion of the ligament. Imbrication of the collagen mesh on to the ligament provides additional tensile strength to the ligament, moreover as the collagen mesh and PMMA microparticles invoke the canine to produce collagen and extracellular matrix, the tensile strength of the ligament at the site of implantation increases. In some embodiments, an implantable device can be imbricated on to the lateral collateral ligament (55) of a canine stifle joint.

Referring to FIG. 4, the canine stifle joint (10) includes the femur (20) and tibia (30). In some embodiments, an implantable device (60) comprising a collagen mesh containing PMMA microparticles can replace the medial collateral ligament of the joint. The implantable device can also include a non-biodegradable substrate, such as a nylon cord, embedded with the collagen mesh containing PMMA microparticles. The collagen mesh and PMMA microparticles invoke the canine to produce collagen and extracellular matrix at the site of replacement, minimizing necrosis at the site of replacement and increasing the tensile strength of the implantable device at the site of replacement. In some embodiments, an implantable device can replace the lateral collateral ligament (55) of a canine stifle joint.

Referring to FIG. 5, the canine stifle joint (10) includes the femur (20) and tibia (30). In some embodiments, the cranial cruciate ligament can be augmented with an implantable device (70) comprising a collagen mesh containing PMMA microparticles. The collagen mesh can be imbricated to at least a portion of the ligament. Imbrication of the collagen mesh on to the ligament provides additional tensile strength to the ligament, moreover as the collagen mesh and PMMA microparticles invoke the canine to produce collagen and extracellular matrix, the tensile strength of the ligament at the site of implantation increases. In more embodiments, an implantable device can be used to replace the cranial cruciate ligament. In such embodiments, the implantable device can also include a non-biodegradable substrate. It is also envisioned that the implantable devices described herein can be used to augment or replace the caudal cruciate ligament (80).

Where the implantable device comprises a gel, paste, liquid or fluid, the device can be delivered to a site at the stifle joint by injection. As will be appreciated, the size of the needle used during such injections will vary according to the subject, viscosity of the implantable device, and application. For example, the needle can have a gauge in the range of about 22 to 25, and length in the range of about 1.5 to 3.0 inches. The volume injected can be less than about 0.1 ml, 0.5 ml, 1.0 ml, 1.2 ml, 1.5 ml, 2.0 ml, 2.5 ml, 5 ml, 10 ml, 20 ml, and 50 ml. Delivery can be to one of more sites at the stifle joint so that the implantable device contacts at least a portion of the stifle joint. Such sites may be intra-articular or extra-articular, and can include the cranial cruciate ligament, caudal cruciate ligament, medial collateral ligament, lateral collateral ligament and capsule. Some embodiments in the form of a gel pr paste are described in U.S. Pat. No. 5,344,452, which is incorporated by reference in its entirety.

Several techniques can be used to guide delivery during injection. Such techniques can include, for example, fluoroscopy, ultrasound, and/or the use of anatomical landmarks only. In preferred embodiments, injections may be with the aid of a fluoroscope. In such embodiments, the implantable device can include a contrast dye to visualize delivery of the implantable device at the site of implantation.

Various modifications to these examples may be readily apparent to those skilled in the art, and the principles defined herein may be applied to other examples without departing from the spirit or scope of the novel aspects described herein. Thus, the scope of the disclosure is not intended to be limited to the examples shown herein but is to be accorded the widest scope consistent with the principles and novel features disclosed herein. Accordingly, the novel aspects described herein is to be defined solely by the scope of the following claims.

1. A method for augmenting the stifle joint in a canine subject comprising:
identifying a subject in need of stifle joint augmentation;
delivering an implantable device to said joint, wherein said implantable device comprises a biodegradable matrix and a plurality of microparticles; and
contacting said implantable device with at least a portion of said joint.

2. The method of claim 1, wherein at least a portion of said joint comprises one or more sites selected from the lateral collateral ligament, medial collateral ligament, cranial cruciate ligament, caudal cruciate ligament, fascia lata, capsule, or patella tendon.

3. The method of claim 1, wherein said implantable device comprises a sheet.

4. The method of claim 2, wherein said sheet comprises fenestrations.

5. The method of claim 1, wherein said biodegradable matrix comprises bovine collagen.

6. The method of claim 1, wherein said biodegradable matrix comprises one or more materials selected from albumin, gelatin, chitosan, hyaluronic acid, starch, cellulose, cellulose derivatives (e.g. methylcellulose, hydroxypropylcellulose, hydroxypropylmethylcellulose, carboxymethylcellulose, cellulose acetate phthalate, cellulose acetate succinate, hydroxypropylmethylcellulose phthalate), casein, dextrans, polysaccharides, fibrinogen, poly(D, L lactide), poly (D, L-lactide-co-glycolide), poly(glycolide), poly(hydroxybutyrate), poly(alkylcarbonate), poly(orthoesters), polyesters, polyhydroxyvaleric acid), polydioxanone, poly(ethylene terephthalate), poly(malic acid), poly(tartronic acid), polyanhydrides, polyphosphazenes, poly(amino acids), and copolymers thereof.

7. The method of claim 1, wherein said plurality of microparticles comprise one or more materials selected from poly methacrylate, polymethyl methacrylate, hydroxapatite, powdered bone, or glass.

8. The method of claim 7, wherein said plurality of microparticles are substantially spherical with a diameter less than 200 μm.

9. The method of claim 8, wherein said plurality of microparticles are substantially spherical with a diameter less than 100 μm.

10. The method of claim 1, wherein said implantable device further comprises a bioactive agent.

11. The method of claim 10, wherein said bioactive agent comprises an agent selected from the group consisting of a local anesthetic, non-steroidal anti-inflammatory drug, antibiotic, and antineoplastic agent.

12. The method of claim 10, wherein said bioactive agent comprises lidocaine.

13. The method of claim 1, wherein said implantable device further comprises a substrate.

14. The method of claim 13, wherein said substrate comprises one or more materials selected from nylon, Dacron, or Teflon.

15. The method of claim 1, further comprising anchoring said implantable device to one or more sites at said joint.

16. The method of claim 1, wherein said delivering comprises an open surgical procedure.

17. The method of claim 1, wherein said delivering comprises percutaneous delivery.

18. The method of claim 1, further comprising performing an extra-articular procedure.

19. The method of claim 18, wherein said extra-articular procedure is selected from fascia lata imbrication, lateral retinacular imbrication, modifications of the lateral retinacular imbrication technique, or posterolateral capsulorrhaphy.

20. The method of claim 1, further comprising performing an intra-articular technique.

21. The method of claim 20, wherein said intra-articular technique is selected from fascia lata repair, modified fascia lata repair, patella tendon repair, or over-the-top repair.

22. The method of claim 1, wherein the cranial cruciate ligament of said subject has undergone partial disruption or fill disruption.

23. A canine stifle joint comprising an implantable device, wherein said implantable device comprises collagen and microparticles.

24. The canine stifle joint of claim 23, wherein said collagen comprises bovine collagen.

25. The canine stifle of claim 23, wherein said microparticles are substantially spherical with a diameter less than 200 μm.

26. The canine stifle of claim 25, wherein said microparticles are substantially spherical with a diameter less than 100 μm.

27. The canine stifle of claim 23, wherein said implantable device further comprises a bioactive agent.

28. The canine stifle of claim 27, wherein said bioactive agent comprises an agent selected from the group consisting of a local anesthetic, non-steroidal anti-inflammatory drug, antibiotic, and antineoplastic agent.

29. The canine stifle of claim 28, wherein said bioactive agent comprises lidocaine.
